(19) United States
(12) Patent Application Publication
(10) Pub. No.: US 2007/0299393 A1
(43) Pub. Date: Dec. 27, 2007

(54) Device and method for surgical treatments

(76) Inventors: Jonathan L. Podmore, San Carlos, CA (US); John E. Crowe, Menlo Park, CA (US)

Correspondence Address: SJM/AFD-WILEY, 14901 DEVEAU PLACE, MINNETONKA, MN, 55345-2126, US

(21) Appl. No.: 11/647,302

(22) Filed: Dec. 29, 2006

Related U.S. Application Data
(60) Provisional application No. 60/815,879, filed on Jun. 23, 2006.

(51) Int. Cl. A61M 29/02; A61B 18/04

(52) U.S. Cl. 604/101.01; 600/192; 606/27

(57) ABSTRACT

The invention relates to an inflatable section on a medical device, which can be expanded or inflated to be precisely placed along or at a desired position in tissue during a medical or surgical procedure. In a preferred embodiment, the inflatable section or sections forms a retracted region, working space, or receiving or docking area for an ablation device, for example during an epicardial ablation procedure where ablation at the pulmonary vein and mitral isthmus regions is desired. Thus, methods and combinations with the devices of the invention can comprise ablation devices and ablation methods, including ablation devices with ultrasound ablation cells.

### CROSS REFERENCE TO RELATED APPLICATIONS

This application claims full priority benefit of U.S. Provisional application 60/815,879, filed Jun. 23, 2006, the entire contents of which are hereby incorporated by reference.

### BACKGROUND OF THE INVENTION

1. Field of the Invention

The invention relates generally to devices and methods used in ablating cardiac and pulmonary vein tissue. In particular, the device and combination of the invention allow safe, beating-heart ablation procedures by providing sufficient operating space for an ablation element position at or near the heart, and/or provide an unobstructed field of view for the physician to precisely place an ablation element. More specifically, devices, combinations, and methods of the invention related to epicardial ablation for the treatment of atrial fibrillation are described, as well as the advantages of devices and methods that position an ablation element at an additional region around the circumferential ablation of pulmonary vein and atrial regions, especially where the additional region is at or near the mitral isthmus.

2. Related Art or Background to the Invention

It is well known that atrial fibrillation results from disorganized electrical activity in the heart muscle, or myocardium. The surgical maze procedure has been developed for treating atrial fibrillation and involves the creation of a series of surgical incisions through the atrial myocardium in a preselected pattern to create conductive corridors of viable tissue bounded by non-conductive scar tissue.

As an alternative to the surgical incisions used in the maze procedure, transmural ablations of the heart have also been used. Such ablations may be performed either from within the chambers of the heart (endocardial ablation) using endovascular devices (e.g., catheters) introduced through arteries or veins, or from outside the heart (epicardial ablation) using devices introduced into the chest. Various ablation techniques have been used, including cryogenic, radiofrequency (RF), laser and microwave. The ablation devices are used to create elongated transmural lesions—that is, lesions extending through a sufficient thickness of the myocardium to block electrical conduction—which form the boundaries of the conductive corridors in the atrial myocardium. Perhaps most advantageous about the use of transmural ablation rather than surgical incisions is the ability to perform the procedure on the beating heart without the use of cardiopulmonary bypass.

In performing the maze procedure and its variants, whether using ablation or surgical incisions, it is generally considered most efficacious to include a transmural incision or lesion that isolates the pulmonary veins from the surrounding myocardium. The pulmonary veins connect the lungs to the left atrium of the heart. Such procedures have been found to offer 57-70% success without antiarrhythmic drugs. However, they are also associated with a 20-60% recurrence rate as the result of lesion recovery, non-pulmonary vein foci of the arrythymia or the need for further tissue modifications.

Previous surgical and catheter-based approaches have demonstrated that linear left atrial (LA) lesions were successful in treating atrial fibrillation when complete block was achieved. One such technique involves linear ablation at the mitral isthmus, defined as extending from the lateral mitral annulus to the ostium of the left inferior pulmonary vein (LIPV). Studies have shown that catheter ablation of the mitral isthmus in combination with pulmonary vein isolation consistently results in demonstrable conduction block and is associated with a high cure rate for paroxysmal atrial fibrillation.

Producing precise lesions at these locations presents significant obstacles for the physician performing endocardial ablations for several reasons. First, while many of the lesions created in the maze procedure can be created from within the right atrium, the pulmonary venous lesions must be created in the left atrium, requiring either a separate atrial access point or a transseptal puncture from the right atrium. Second, the elongated and flexible endovascular ablation devices are difficult to manipulate into the complicated geometries required for forming the pulmonary venous lesions. Maintaining the proper positioning against the wall of a beating heart is also difficult. Third, visualization of endocardial anatomy and endovascular devices is often inadequate and knowing the precise position of such devices in the heart can be difficult, resulting in misplaced lesions.

Epicardial ablation devices and methods useful for creating transmural lesions for the treatment of atrial fibrillation have been described in U.S. Pat. No. 7,052,493 to Vaska, et. al. and U.S. Pat. No. 6,971,394 to Sliwa, et. al., each incorporated by reference into this application. Sliwa describes a method of forming a transmural lesion in a wall of the heart adjacent to the pulmonary veins by placing an ablation device through a thoracic incision and then through a pericardial penetration so that the ablation device is disposed in contact with an epicardial surface of the heart. Vaska describes an ablation device and system which may be used to wrap an ablation device around the pulmonary veins at an epicardial location.

In order to take full advantage of the synergistic benefits of combining linear left atrial ablations (like the mitral isthmus ablation) with PV isolation, it is important that these ablations have continuity with one another. Failure to provide this continuity may allow for reentry pathways, which limit the effectiveness of the treatment. However, execution of a contiguous mitral isthmus ablation following PV isolation presents considerable challenges to the physician. Difficulties in visualizing the precise location of a pre-existing PV-isolation ablation compounded with the challenges of maintaining accurate placement on a beating heart mean that a high degree of physician skill and experience are required in order to repeatedly create contiguous ablations.

What are needed, therefore, are devices and methods that allow for the safe and precise placement of ablation elements and which insure continuity of the PV-isolation ablation with a mitral isthmus ablation. The devices, combinations, and methods of the invention preferably allow the physician to deploy ablation devices in a precise manner by minimizing the extent to which anatomical obstructions block the physicians view of the target tissue and assist the physician with precise placement of ablating devices on the beating heart.

### BRIEF SUMMARY OF THE INVENTION

The present invention meets these and other objectives by providing devices and methods for placing ablation elements on epicardial surfaces, especially during the mitral isthmus ablation of an atrial fibrillation treatment. The present invention can be integrated with existing PV-isolation devices, allowing for better continuity of additional ablations in minimally invasive approaches. In one aspect, the invention comprises an inflatable section which can be precisely placed along the mitral isthmus to assist the physician with the placement of the ablating device. In preferred embodiments, a combination of devices of the invention comprises an ultrasound ablation element, such as a piezoelectric cell as described in U.S. Pat. No. 7,052,493, incorporated herein by reference in its entirety. Various other ablation elements, devices, or techniques can be used, including cryogenic ablation, radiofrequency (RF) ablation, laser ablation, microwave ablation, and biological agent delivery systems.

In another aspect, the inflatable sections of the invention retract obstructions from the physician's field of view, allowing visual confirmation of correct placement of the device. Retracting obstructions also creates sufficient working space for the ablation device, which adds to the safety of the ablation procedure by helping to prevent ablation damage to adjacent tissues.

In another aspect, the invention comprises a method of ablating tissue, especially heart tissue, comprising wrapping an elongate, flexible device around an anatomical structure of the heart. The elongate device includes at least one inflatable section whose location on the device is designed to correlate closely with the mitral annulus on the heart. Here and elsewhere in this description, the term “inflatable” preferably refers to an element than can be filled with air, gas, or fluid, but the term can additionally be an element that expands by mechanical forces or devices to fill an increased volume or area when in operation.

In an advantageous aspect of the invention, the inflatable sections can be deployed in the same manner as the primary ablating device around the PV isolation line. Thus, the sections are essentially automatically placed into a contiguous position with the PV-isolation ablation lines to form an effective additional ablation line toward the mitral isthmus. This results in improved and more reproducible treatment of atrial fibrillation. Furthermore, by deploying the sections in the same manner as the primary PV-isolation device, the invention is also able to use the same access to the epicardial tissue.

While ultrasound ablation devices are preferred, the invention can be used with any type of or any available ablation device, treatment device, or ablation element suitable for use on human tissue. Combinations of ablating devices or ablating elements can also be used.

### BRIEF DESCRIPTION OF THE DRAWINGS

FIG. 1 depicts a top (B), side (C) and isometric (A) view of an inflatable section with a U-shape, with an ablation device shown positioned in the center of the inflated region in B.

FIG. 2 depicts a top (B), side (C) and isometric (A) view of another U-shaped inflatable section thinner and with a shallower height than in FIG. 1.

FIG. 3 depicts a top (B), side (C) and isometric (A) view of an inflatable section with an L-shape.

FIG. 4 depicts a top (A), side (C) and isometric (B) view of another L-shaped inflatable section thinner and with a shallower height than in FIG. 3.

FIG. 5 depicts a top (B), side (C) and isometric (A) view of an L-shaped inflatable section including roof channels.

FIG. 6 depicts a top (B), side (C) and isometric (A) view of another L-shaped inflatable section embodiment having a roof channels and a deeper height or larger profile than that shown in FIG. 5.

FIG. 7 schematically depicts an ablation devise positioned on an epicardial surface at an inflated region of a device of the invention.

### DETAILED DESCRIPTION OF THE INVENTION

The headings (such as “Brief Summary”) used are intended only for general organization of topics within the disclosure of the invention and are not intended to limit the disclosure of the invention or any aspect of it. In particular, subject matter disclosed in the “Related Art” includes aspects of technology within the scope of the invention and thus may not constitute solely background art. Subject matter disclosed in the “Brief Summary” is not an exhaustive or complete disclosure of the entire scope of the invention or any particular embodiment.

As used herein, the words “preferred,” “preferentially,” and “preferably” refer to embodiments of the invention that afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention and no disclaimer of other embodiments should be inferred from the discussion of a preferred embodiment or a figure showing a preferred embodiment. Also, this disclosure refers to various inflatable sections or an inflatable section. In each case, the use of the singular or plural terms is merely optional and in each case the singular may be replaced by the plural and vice versa.

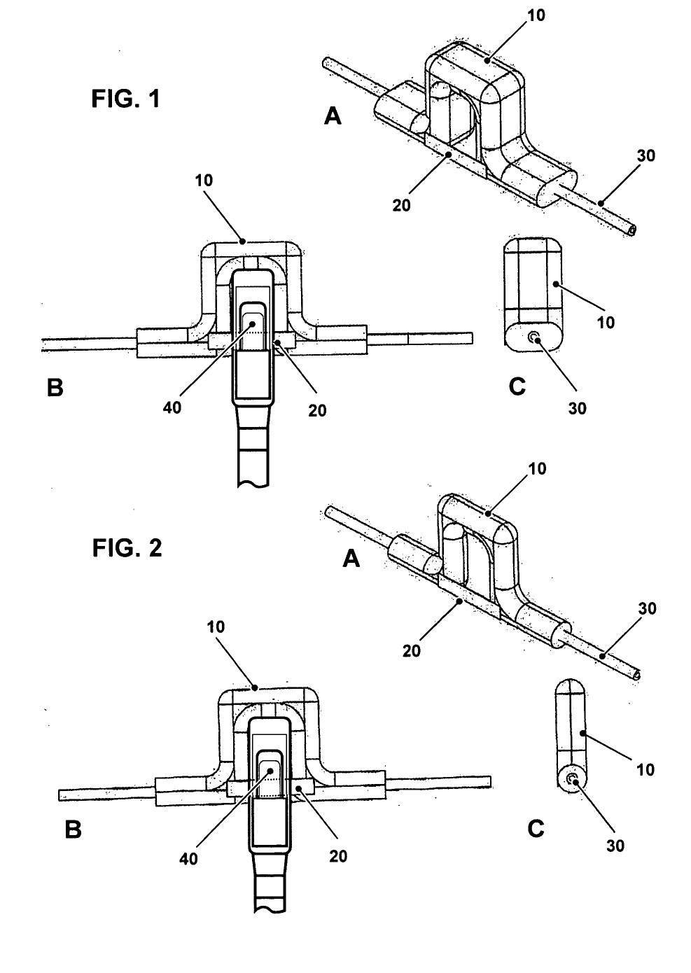

FIG. 1A-C depict a first embodiment of the present invention. In this embodiment, an inflatable section 10 is shown having a U-shape. The U-shaped inflatable section creates a slot which can act as a guide for positioning the ablation elements of a catheter or other ablation device. A structural member 20 optionally rests on a side of the inflatable section and bridges the slot which helps the inflatable section to maintain its shape. The inflatable sections may inflate by the addition of a gas, air, or fluid, or by a mechanical expansion of the outside walls through moving rods, plates, screws, or similarly actuated elements. The outside walls can be partially or fully supported by structural elements, or simply be an inflatable, biocompatible polymer material acting like a balloon. Channels 30 on either side of the inflatable section can deliver the air, gas, or fluid to expand and deflate the inflatable sections. Channel 30 can also or in addition be a conduit for a wire or other connection to the mechanical movement device that expands the inflatable section at the beginning of a procedure, and then can deflate the section when the procedure is completed.

In FIGS. 1-4, an embodiment is shown where the inflatable sections are essentially separate compartments into which air, gas, or a fluid can be introduced to and removed from. These inflatable sections can be soft, polymeric tubes prepared to a desired size that are operably connected with at least one hole between each set to allow the gas, air, or fluid to fill all of the sections. In a preferred method, the polymeric tubes are RF welded together to insure proper sealing. Similarly, each tube section can be loaded with a mechanical device that expands the volume of the tube when actuated. In alternative embodiments, the inflatable sections can each be separately inflated or expanded, or desired combinations of section can be inflated or expanded together.

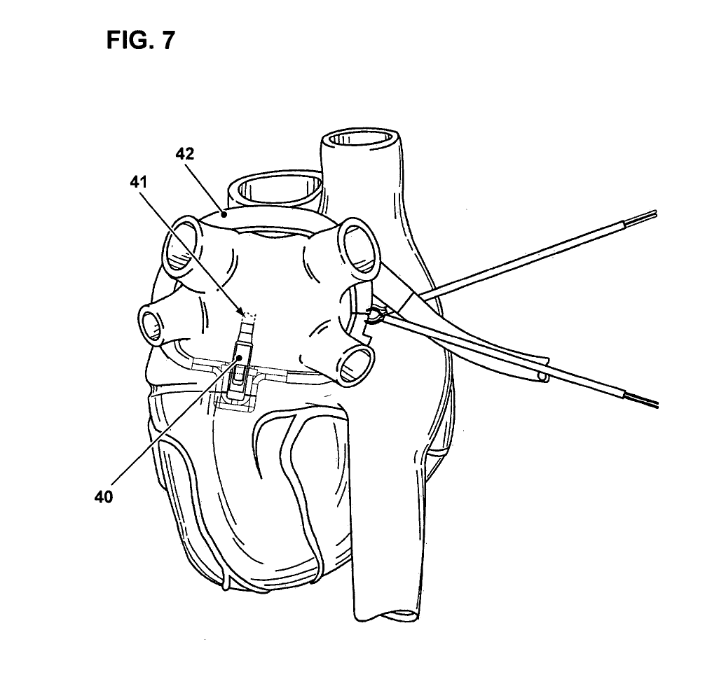

In treatments of cardiac tissue as shown in the FIG. 7 example, after placement between the pericardium and the epicardial tissue or where ablation is desired, the inflatable section can be inflated or expanded to make room for the insertion or docking of ablation device 40. As the inflatable section expands it creates additional space between the pericardium and epicardium into which an ablation device 40 and/or other instruments can be worked. The U-shape of the inflatable section also provides a slot or receiving or docking area, which provides the physician with a precise location into which the ablation device 40 can be placed. Thus, the device of the invention can be used to help position an ablating or treatment device over a desired or target area. In the example of FIG. 7, the circumferential ablation of atrial tissue can be performed over the area covered by band 42, and once the ablation elements are removed, the band 42 with inflatable sections can provide a receiving area for ablating the mitral isthmus, for example. Similarly, band 42 can comprise ablation elements along its length and the additional inflatable sections at a desired position. In FIG. 7, ablating device 40 has a optionally flexible shaft 41 so that the mitral isthmus can be introduced into the receiving area below the inferior pulmonary vein. In a preferred example, the invention can include a kit having an ablation device with angled shaft 41, for example about 90 degrees, or between about 45 degrees and about 100 degrees, compared to the shaft region connection to the relatively flat or slightly curved ablating surface on the ablating device 40. While the specific examples discussed here include atrial fibrillation ablations, the invention and its use are not limited to cardiac tissue and atrial fibrillation treatments.

FIG. 2A-C depict a second embodiment of the invention. In this embodiment, a U-shaped inflatable section is also shown but is designed to inflate to a shallower height and have a lower profile than the embodiment of FIG. 1, which illustrates that more or less retracting capability can be built into the inflatable sections for ablation devices and other instruments.

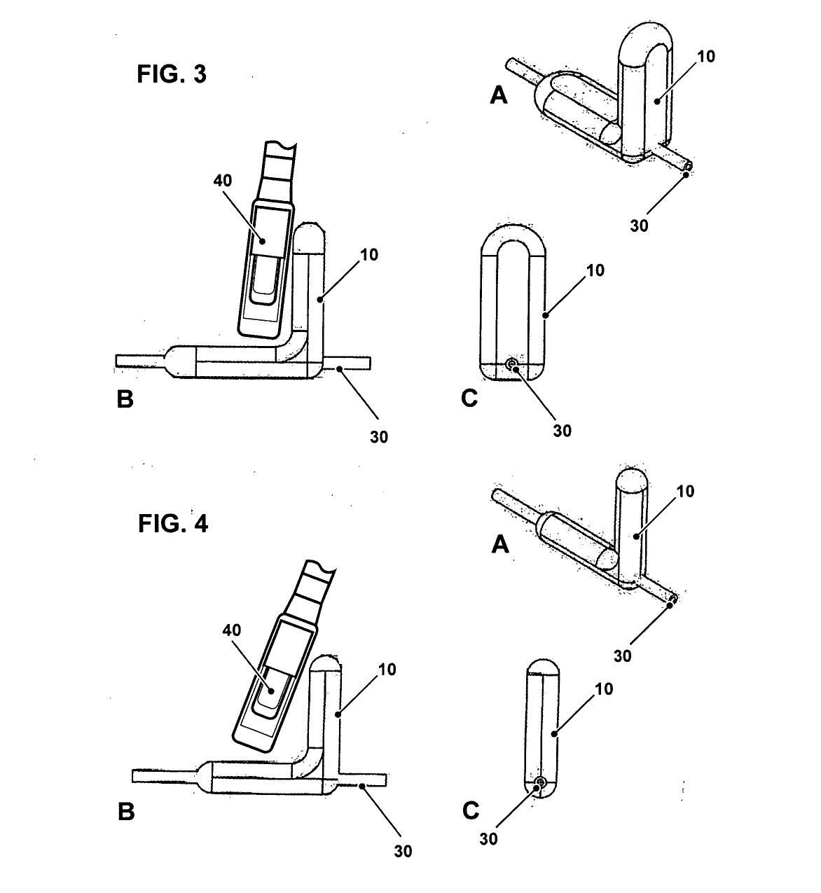

FIG. 3A-C depict a third embodiment of the invention. In this embodiment, the inflatable section 10 is fashioned in an L-shape. The L-shape is formed by at least two inflatable sections configured to be approximately orthogonal to each other, or about 90 degrees apart, when inflated or expanded. Various other angles between the two or more inflatable sections to create the L-shape can also be selected and used. The L-shaped inflatable sections will also create a working space between the pericardium and epicardium when inflated or expanded. The L-shape of this embodiment does not create a confined slot and is therefore more appropriate for situations where the physician desires more freedom to move the ablation device 40 or other instruments. Similarly, FIG. 4A-C depict a fourth embodiment, which also features the L-shaped inflatable sections. The inflatable sections of FIG. 4 are designed to inflate to a shallower height, which illustrates that more or less retracting capability can be built into the inflatable sections for ablation devices and other instruments.

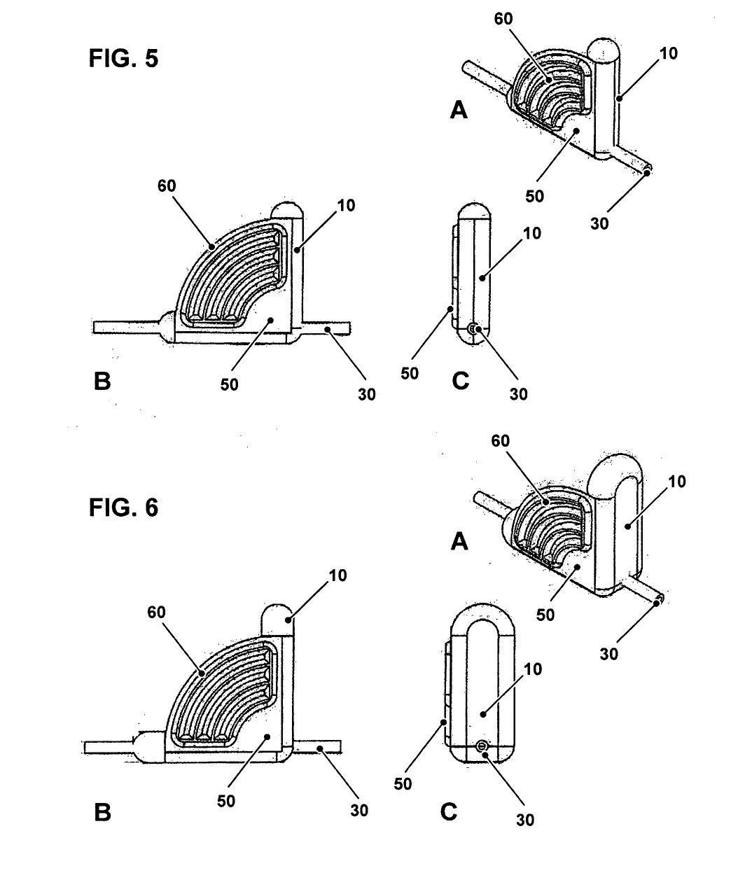

FIG. 5A-C depict a fifth embodiment of the invention. In this embodiment, the inflatable section 10 is once again fashioned in an L-shape. The advantages of freedom of movement for the ablating device will also be realized in this embodiment. FIG. 5 also includes a roof 50, which extends over the top of the L-shaped inflatable sections. The roof 50 further comprises a series of channels or inflatable channels 60 which run along the surface of the roof. The roof is meant to provide additional, unobstructed space for the ablation device or any accompanying devices to work in, or to assist in viewing the placement of ablation or treatment devices. It is understood that in some instances where the L-shaped inflatable sections could be relatively large that portions of the pericardium resting above the inflatable section could “sag” into the working space within the legs of the inflatable section. A roof section helps to keep the pericardium retracted in these spaces to allow maximum working and viewing space. The addition of the inflatable channels 60 along the top of the roof can provide the physician with even greater flexibility by further retracting the pericardium if inflated. FIG. 6A-C depict a sixth embodiment, which illustrates that more or less retracting capability can be built into the inflatable section by varying the height it can achieve during inflation. The inflated section 10 of FIG. 6 is raised or has a larger profile in comparison to the same section 10 in FIG. 5. In these and all embodiments of the inflatable section or sections, and combinations of sections forming shapes or docking regions, each inflatable section can be either individually controlled and inflated or multiple sections can be inflated together. In addition, each section can be fully inflated or inflated less than fully, and different combinations of multiple sections may be fully inflated while others are not inflated or are not fully inflated.

The inflatable sections can be introduced into the epicardial space by any suitable device or method. One such device is described in U.S. Pat. No. 7,052,493 (specifically incorporated herein by reference) to Vaska, et. al. Vaska describes devices and methods for introducing and placing elongated ablation devices which encircle the pulmonary veins of the heart. By placing an inflatable device on an elongate, flexible section like the one described in Vaska, the placement of the inflatable device can be accurately directed to a location contiguous with a previous PV-isolation ablation or lesion. This has the advantage of placing the inflatable sections into the same position as the PV-isolation ablation lines and thus allows for the formation of an effective additional ablation line, for example extending continuously to the mitral isthmus, to result in improved treatment of atrial fibrillation. By deploying in the same manner as the PV-isolation device, the devices and methods of the invention are also able to use the same access to the epicardial tissue site.

Finally, although the present methods and devices have been described in connection with cardiac tissue, the invention is not limited to a particular treatment or use. A preferred example is creating additional ablation regions at or coincident with a contiguous, circumferential lesion around the pulmonary veins and/or atrial region of the heart. It is understood that the methods are equally applicable for non-contiguous lesions, or any lesions around other anatomical features. Furthermore, other lesions may be beneficial in treating electrophysiological conditions and the devices and methods described herein may be useful in creating such other lesions. Thus, the present invention should not be construed as being limited to creating contiguous lesions with those around the pulmonary veins.

Although embodiments of this invention have been described above with a certain degree of particularity, those skilled in the art could make numerous alterations to the disclosed embodiments without departing from the spirit or scope of this invention. For example, although the invention is described as using ablation elements that are deployed on a separate device it would be possible to combine the inflatable sections directly onto an ablating device.

All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the invention as defined in the appended claims.

What is claimed is:

1. An inflatable medical device or part thereof, comprising an elongate flexible body and at least a first and second inflatable sections incorporated into the body, wherein the inflatable sections are connected and can be filled or expanded at the same time or through the same operation, and the first section inflates or expands along the axis of the elongate flexible body and the second section inflates or expands in a direction outside of the axis of the elongate flexible body.

2. The inflatable medical device of claim 1, wherein the second section expands or inflates in an orthogonal direction compared to the elongate flexible body.

3. The inflatable medical device of claim 2, further comprising an additional inflatable section, which, in combination with the first and second sections, forms a U-shaped region when all sections are inflated or expanded.

4. The inflatable medical device of claim 2, further comprising a roof section that deploys between the first and second inflated or expanded sections.

5. The inflatable medical device of claim 1, wherein the sections are composed of a biocompatible polymer.

6. The inflatable medical device of claim 1, wherein the sections are connected by a fill tube to a supply line for air, gas, or a fluid.

7. The inflatable medical device of claim 2, wherein the sections are connected by a fill tube to a supply line for air, gas, or a fluid.

8. The inflatable medical device of claim 3, wherein the sections are connected by a fill tube to a supply line for air, gas, or a fluid.

9. The inflatable medical device of claim 1, wherein a mechanical device operates to expand the sections to an inflated volume.

10. A medical device having a positioning region for use in ablating tissue, the device comprising an elongate flexible body with an inflatable section to form a receiving area for one or more ablating elements when inflated.

11. The medical device of claim 10, wherein the receiving area is sized to cover an area at or near the human pulmonary vein-left atrial region of the heart.

12. The medical device of claim 11, wherein the receiving area is designed to cover the mitral isthmus region.

13. The medical device of claim 10, wherein multiple inflatable sections are employed to form a receiving area.

14. A method for positioning an ablating device comprising introducing an inflatable device to a target tissue area, inflating the inflatable device to form a receiving area of retracted tissue, and introducing and ablation device into the receiving area.

15. The method of claim 14, wherein the target tissue area is at or near the human pulmonary vein-left atrial region of the heart.

16. The method of claim 14, wherein the receiving area is formed by multiple inflatable sections.

17. The method of claim 16, wherein at least one of the multiple inflatable sections is fully inflated.

18. A method for retracting tissue during an epicardial treatment procedure, comprising providing a medical device of claim 1 at the surface of the heart and inflating the device.

19. A method for retracting tissue during an epicardial treatment procedure, comprising providing a medical device of claim 2 at the surface of the heart and inflating the device.

20. A method for retracting tissue during an epicardial treatment procedure, comprising providing a medical device of claim 3 at the surface of the heart and inflating the device.

21. A method for retracting tissue during an epicardial treatment procedure, comprising providing a medical device of claim 4 at the surface of the heart and inflating the device.
